(19) United States
(12) Patent Application Publication
(10) Pub. No.: US 2023/0147710 A1
(43) Pub. Date: May 11, 2023

(54) INDIVIDUALIZED LIQUID FEEDING

(71) Applicant: Canine Life Sciences, Inc, Denver, CO (US)

(72) Inventors: Korinn E. Saker, Hillsborough, NC (US); Jeff Sutherland, Aurora, CO (US)

(21) Appl. No.: 18/053,008

(22) Filed: Nov. 7, 2022

Related U.S. Application Data
(60) Provisional application No. 63/276,728, filed on Nov. 8, 2021.

(51) Int. Cl. G16H 20/60

(52) U.S. Cl. CPC G16H 20/60

(57) ABSTRACT

A system for individualized liquid feeding includes a powdered protein, a nutritional oil, a powdered, low fat, low protein food fortified with vitamins and minerals and a computer program product for individualized liquid feeding. The computer program product is configured to cause a computing device to perform a number of actions. According to one action, the computing device, based in part upon an input mass, computes a suitable daily amount of the powdered, low protein, low fat food. According to another action, the computing device, based in part upon an input primary health condition, computes an amount of the supplemental powdered protein and an amount of the nutritional oil to be combined with the amount of the powdered, low protein, low fat food. According to another action, the computing device outputs the computed amounts of the powdered, low protein, low fat food, the supplemental powdered protein and the nutritional oil.

### CROSS-REFERENCE TO RELATED APPLICATIONS

This application claims the priority benefit of U.S. Provisional Application No. US 63/276,728 filed on 8 Nov. 2021, pending, which is incorporated herein by reference in its entirety.

### SUMMARY

The disclosure describes a system for individualized liquid feeding. The system includes a powdered protein, a nutritional oil, a powdered, low fat, low protein food fortified with vitamins and minerals and a computer program product for individualized liquid feeding. The computer program product includes a non-transitory computer-readable storage medium storing computer-executable code for outputting liquid feeding instructions. The code, when executed, is configured to cause a computing device to perform a number of actions. According to one action, the computing device is caused to, based in part upon an input mass, compute a suitable daily amount of the powdered, low protein, low fat food. According to another action, the computing device is caused to, based in part upon an input primary health condition, compute an amount of the supplemental powdered protein to be combined with the amount of the powdered, low protein, low fat food. According to another action, the computing device is caused to, based in part upon the input primary health condition, compute an amount of the nutritional oil to be combined with the amount of the powdered, low protein, low fat food. According to another action, the computing device is caused to output the computed amount of the powdered, low protein, low fat food, the computed amount of the supplemental powdered protein and the amount of the nutritional oil.

The disclosure also describes another system for individualized liquid feeding of an individual. The system includes a sealed vessel containing a powdered protein, a sealed vessel containing a nutritional oil, a sealed vessel containing a powdered, low fat, low protein food fortified with vitamins and minerals as well as instructions for mixing a portion of the powdered, low fat, low protein food with a portion of the powdered protein and a portion of the nutritional oil in accordance with a mass and primary health condition of the individual.

Further, the disclosure also describes a method for individualized liquid feeding of an individual. The method includes providing a sealed container of a powdered, low fat, low protein food fortified with vitamins and minerals together with a sealed container of a powdered protein together with a sealed container of a nutritional oil and providing instruction to combine a portion of the powdered, low fat, low protein food with a portion of the powdered protein and a portion of the nutritional oil in accordance with a mass and primary health condition of the individual.

### BRIEF DESCRIPTION OF THE FIGURES

The summary above, as well as the following detailed description of illustrative embodiments, is better understood when read in conjunction with the appended drawings. For the purpose of illustrating the disclosure, example constructions are shown in the drawings. However, the disclosure is not limited to specific methods and instrumentalities disclosed herein. Moreover, those having ordinary skill in the art will understand that the drawings are not to scale. Wherever possible, like elements have been indicated by identical numbers.

Embodiments of the disclosure will now be described, by way of example only, with reference to the following diagrams wherein:

FIG. 1 illustrates an example method for preparing a formula for individualized liquid feeding.

FIG. 2 illustrates example alternative methods for individualized liquid feeding.

FIG. 3 schematically illustrates an example network environment suitable for practicing disclosed systems and methods.

FIG. 4 schematically illustrates example components of an example computing device suitable for practicing disclosed systems and methods.

FIG. 5 illustrates an example user interface suitable for use in association with disclosed methods and systems.

FIG. 6 illustrates an example user interface suitable for use in association with the user interface of FIG. 5 and in association with disclosed methods and systems.

FIG. 7 illustrates an example user interface for use in association with the user interfaces of FIGS. 5 & 6 and in association with disclosed methods and systems.

FIG. 8 illustrates an example user interface presenting example computed amounts of formula ingredients.

### DETAILED DESCRIPTION

The following detailed description illustrates embodiments of the disclosure and manners by which they can be implemented. Although the preferred mode of carrying out the present disclosure has been disclosed, those skilled in the art would recognize that other embodiments for carrying out or practicing the present disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

Some individuals including humans and animals such as cats and dogs may receive their nutrition via enteral feeding tube or oral syringe which necessitates a liquid form. Known nutrition systems require a caregiver or other user separately calculate an individual's Resting Energy Requirement (RER) in kilocalories, to look up various conditions and their associated dietary profiles (such as protein and fat percentages) and make a ‘best-fit’ approximation for an individual's health condition. Further, known systems have a fixed ratio of macronutrients and do not enable varying fat or protein levels. There are also logistical costs and challenges with pre-mixed liquids.

Embodiments of the disclosure substantially eliminate, or at least partially address, problems in the prior art, enabling determination of an optimal diet profile of multiple components based on disease state(s) and physical characteristics for each patient or other individual being fed.

Additional aspects, advantages, features and objects of the disclosure will be made apparent from the drawings and the detailed description of the illustrative embodiments construed in conjunction with the appended claims that follow.

It will be appreciated that described features are susceptible to being arranged in various combinations without departing from the scope of the disclosure as defined by the appended claims.

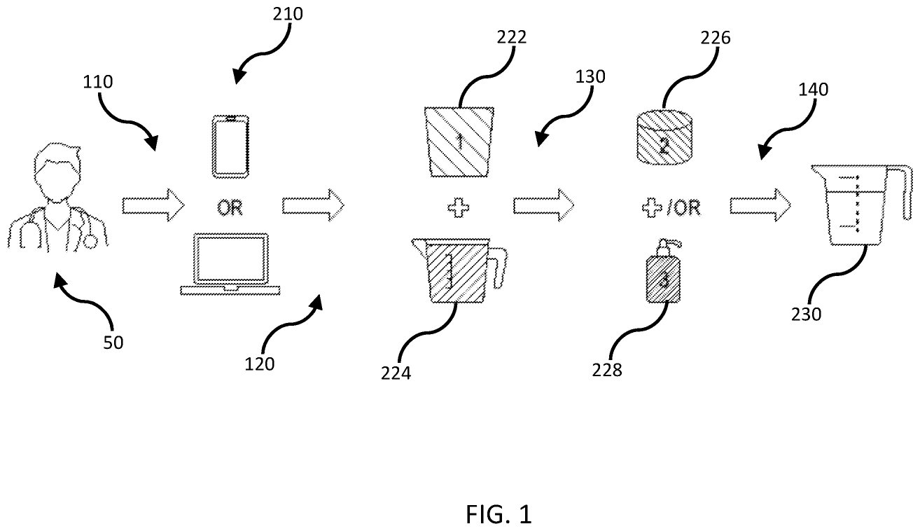

FIGS. 1 & 2 illustrate an example method for individualized liquid feeding. The method, which may be partially computer-implemented or computer-supported, represents a collection or sequence of actions that may be implemented in hardware, software, or a combination thereof. A powdered food having low fat content, low protein content, vitamins and minerals is supplied along with a powdered protein and a nutritional oil. Each of the low fat, low protein powdered food, the powdered protein and the nutritional oil may be provided in separate, sealed packaging to facilitate customized formulas.

Further, the method includes providing instruction to combine a portion of the powdered, low fat, low protein food with a portion of the powdered protein and a portion of the nutritional oil in accordance with a mass and primary health condition of the individual.

An amount of the powdered, low protein, low fat food is computed based at least in part upon a mass of an individual. Based upon a primary health condition of the individual, a ratio of nutrients is determined and, from this ratio, amounts of the supplemental powdered protein and nutritional oil to be combined with the powdered, low protein, low fat food is identified or computed.

For example, from a mass and a primary health condition at 110, a clinician 50 may compute a suitable amount of the powdered, low protein, low fat food. An amount of the supplemental powdered protein and an amount of the nutritional oil to be combined with the powdered, low protein, low fat food may be computed with consideration for at least a primary health condition. In an example, a moderate fat profile and moderate protein profile may be suitable for an individual with an allergy while a high fat profile and moderate protein profile may be suitable for an individual with cancer while a moderate fat profile and a low protein profile may be suitable for an individual with Chronic Kidney Disease stage 3 or 4.

The clinician may compute amounts and/or use instruction provided to a computing device 210 such as a desktop computer, laptop computer, tablet computer or mobile phone or may compute amounts using instructions provided in printed form and/or electronic ink.

The result of the computations is output at 120 and the computed amount of powdered, low protein, low fat food 222 is measured out for hydrating with an amount of water 224 at 130. Further benefits of the powdered, low protein, low fat food include but are not limited to a low phosphorous content. The powdered, low protein, low fat food may also include one or more emulsifiers such as lecithin.

In an example, the powdered, low protein, low fat food is comprised of 8% fat and 16% protein on a dry matter basis. In a further example, the powdered, low protein, low fat food is comprised of 0.37% Calcium and 0.12% phosphorus on a dry matter basis. Such formulations may be suitable for a canine species, for example.

In another example, the powdered, low protein, low fat food is comprised of 30% fat and 35% protein on a dry matter basis. In a further example, the powdered, low protein, low fat food is comprised of 0.5% Calcium and 0.2% phosphorus. In a still further example, the powdered, low protein, low fat food is comprised of 0.25% taurine on a dry matter basis. Such formulations may be suitable for a feline species, for example.

An amount of water to be combined with the powdered, low protein, low fat food is computed based upon a desired consistency or a desired viscosity of the liquid formula. For example, the powdered food is sufficiently hydrated such that it can be pushed through a 3 FR lumen at 0.2 ml/s. At 140, the hydrated amount of the powdered food is combined with powdered protein 226 and/or nutritional oil 228 to yield a complete liquid formula 230 suitable for liquid feeding.

Powdered protein 226 may include a blend of amino acids and minerals. Nutritional oil 228, which is a suitable source of eicosapentaenoic acid (EPA), docosahexaenoic acid (DHA) and/or omega 3 PUFAs (polyunsaturated fatty acids), may be a marine oil or a plant oil. In an example, the nutritional oil is salmon oil.

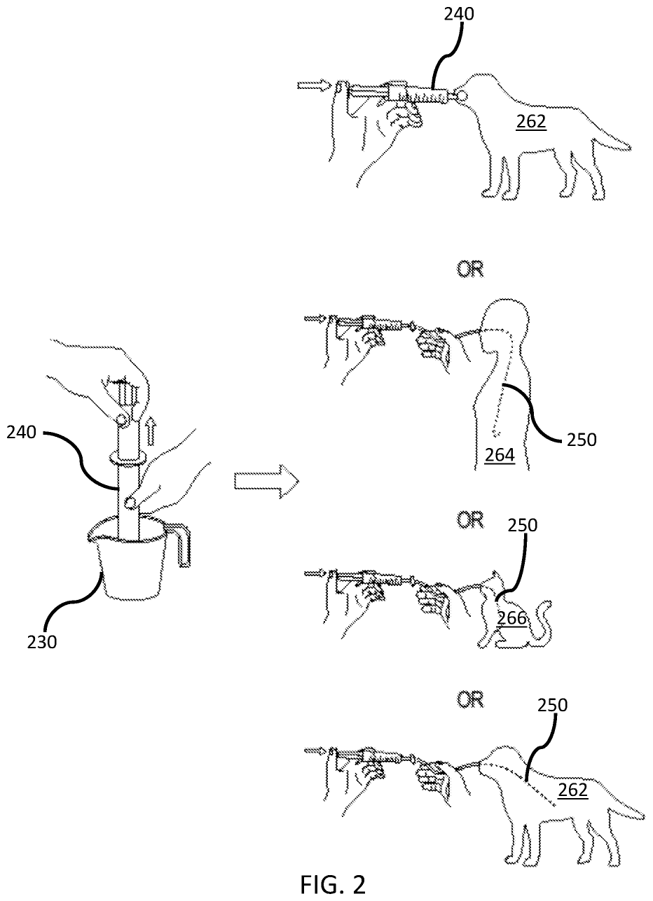

Referring to FIG. 2, prepared formula 230 is drawn into a syringe 240 and may be fed to the individual directly with the syringe or through an enteral feeding tube 250. Formula 230 may be fed to any of a variety of individuals including but not limited to individual canines 262, individual humans 264 or individual felines 266.

The actions described above are only illustrative and other alternatives can also be provided where one or more actions are added, one or more actions are removed, or one or more actions are provided in a different sequence without departing from the scope of the claims herein.

Above-described methods for individualized liquid feeding may be practiced in association with any of a variety of systems. An example system for individualized liquid feeding of an individual includes a sealed vessel containing a powdered protein, a sealed vessel containing a nutritional oil, a sealed vessel containing a powdered, low fat, low protein food fortified with vitamins and minerals as well as instructions for mixing a portion of the powdered, low fat, low protein food with a portion of the powdered protein and a portion of the nutritional oil in accordance with a mass and primary health condition of the individual. The instructions may be provided as graphical user interface output.

The graphical user interface may be output in association with a computer program product for individualized liquid feeding. The computer program product includes a non-transitory computer-readable storage medium storing computer-executable code for outputting liquid feeding instructions. The code, when executed, is configured to cause a computing device to perform a number of actions associated with above-described methods.

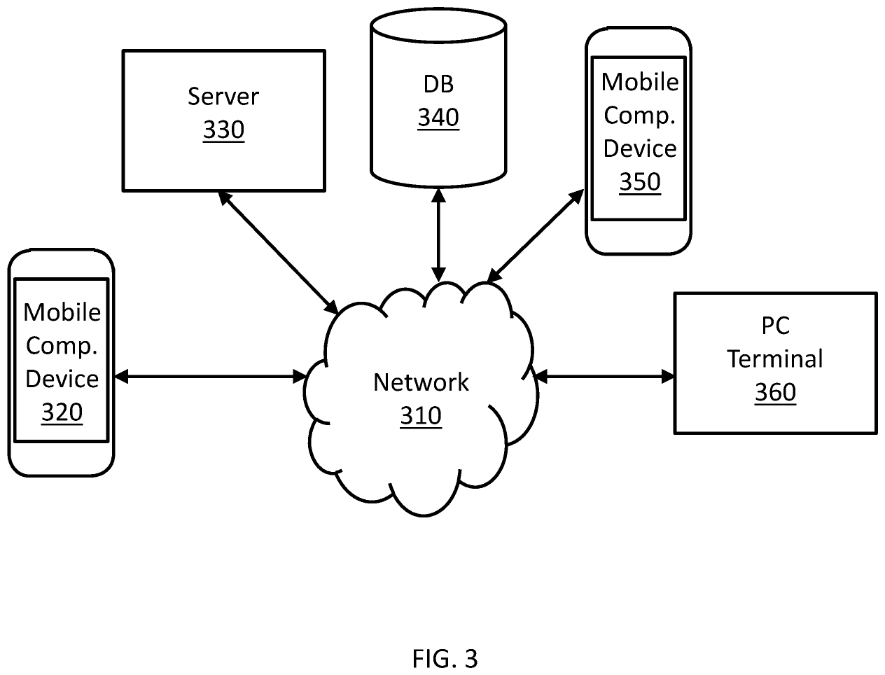

FIG. 3 schematically illustrates an example network environment that is suitable for practicing embodiments of the disclosure. Network environment 300 includes one or more mobile computing devices 320, a PC Terminal 360, a server 330, a database 340 associated with server 330, and a communication network 310.

Network environment 300 may be implemented in various ways, depending on various possible scenarios. In one example scenario, network environment 300 may be implemented by way of a spatially collocated arrangement of server 330 and database 340. In another example scenario, network environment 300 may be implemented by way of a spatially distributed arrangement of server 330 and database 340 coupled mutually in communication via communication network 310. In yet another example scenario, server 330 and database 340 may be implemented via cloud computing services.

Mobile computing devices 320 and 350 and PC terminal 360 are coupled in communication with server 330 via communication network 310. Communication network 310 can be a collection of individual networks, interconnected with each other and functioning as a single large network. Such individual networks may be wired, wireless, or a combination thereof. Examples of such individual networks include, but are not limited to, Local Area Networks (LANs); Wide Area Networks (WANs); Metropolitan Area Networks (MANs); Wireless LANs (WLANs); Wireless WANs (WWANs); Wireless MANs (WMANs); the Internet; second, third, fourth or fifth generation (5G) telecommunication networks; and Worldwide Interoperability for Microwave Access (WiMAX) networks.

Examples of mobile computing devices 320 and 350 and/or PC terminal 360 include, but are not limited to, mobile phones, smart telephones, Mobile Internet Devices (MIDs), tablet computers, Ultra-Mobile Personal Computers (UMPCs), phablet computers, Personal Digital Assistants (PDAs), web pads, Personal Computers (PCs), handheld PCs, laptop computers and desktop computers.

Network environment 300 is suitable for implementing various systems for individualized liquid feeding. In order to implement a system for individualized liquid feeding, server 330 provides an individualized liquid feeding service to mobile computing devices 320 and 350 and/or PC terminal 360, while database 340 stores data related to the service.

Mobile computing devices 320 and 350 and/or PC terminal 360 may access server 330 to download one or more software products associated with the individualized liquid feeding service. In one embodiment, the system is arranged in a manner that its functionality is implemented partly in mobile computing devices 320 and 350 and/or PC terminal 360 and partly in server 330. For example, the service may be browser-based. In another embodiment, the system is arranged in a manner that its functionality is implemented entirely in mobile computing devices 320 and 350 and/or PC terminal 360.

Users associated with mobile computing devices 320 and 350 and/or PC terminal 360 use the individualized liquid feeding service. Accordingly, mobile computing devices 320 and 350 and/or PC terminal 360 may be used, operated, or carried by clinicians, legal guardians or handlers of an individual or individuals requiring or benefiting from liquid feeding.

In an embodiment of the disclosure, mobile computing devices 320 and 350 and PC terminal 360 and/or server 330 substantially continuously record and update changes in the status of individual or individuals in database 340 based on input by the user. The status of the individual or individuals in the database may, for example, include health status and associated time-stamps. Furthermore, the individualized liquid feeding service can be offered free of cost. Alternatively, the service can be a paid service that has a subscription-based billing or a transaction-based billing, such as pay-per-use and pay-per-feature.

FIG. 3 is merely an example, which should not unduly limit the scope of the claims herein. It is to be understood that the specific designation for network environment 300 is provided as an example and is not to be construed as limiting the network environment to specific numbers, types, or arrangements of mobile computing devices 320 and 350, PC terminals 360, servers 330, databases 340 and communication networks 310. A person of ordinary skill in the art will recognize many variations, alternatives, and modifications of embodiments of the disclosure.

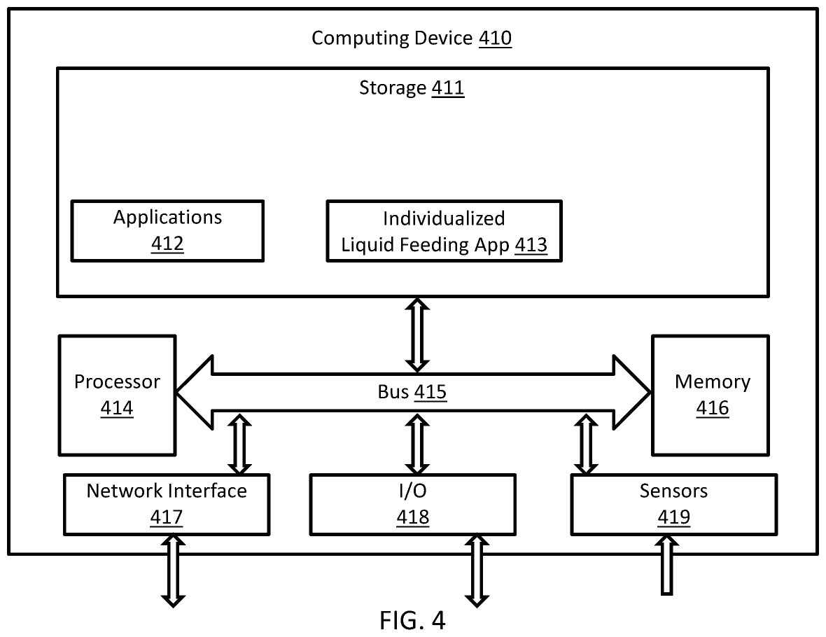

FIG. 4 is a schematic illustration of various components of a computing device 410, in accordance with an embodiment of the disclosure. Computing device 410 could be implemented as the mobile computing devices 320 or 350 or PC terminal 360 of FIG. 3, and vice versa and is configured for use in association with disclosed individualized liquid feeding services and systems for individualized liquid feeding. Further, computing device 410 may be configured for use in association with server 330 for performing a method for individualized liquid feeding.

Computing device 410 includes, but is not limited to, a memory 416, a computing hardware such as a processor 414, Input/Output (I/O) devices 418, a network interface 417, a configuration of sensors 419, a storage 411, and a system bus 415 that operatively couples various components including memory 416, processor 414, I/O devices 418, network interface 417, sensors 419 and storage 411. I/O devices 418 include a display screen for presenting graphical images to a user of computing device 410. Computing device 410 also includes a power source for supplying electrical power to the various components. The power source may, for example, include a rechargeable battery.

In some examples, the display screen may be a touch-sensitive display screen that is operable to receive tactile inputs from the user. These tactile inputs may, for example, include clicking, tapping, pointing, moving, pressing and/or swiping with a finger or a touch-sensitive object like a pen.

Additionally or alternatively, I/O devices 418 may also include a microphone for receiving an audio input from the user, and a speaker for providing an audio output to the user.

Memory 416 optionally includes non-removable memory such as Random-Access Memory (RAM) or Read-Only Memory (ROM). Storage 411 optionally includes non-removable memory, removable memory, or a combination thereof. Non-removable memory of storage 411, for example, includes flash memory, or a hard drive. Removable memory of storage 411, for example, includes flash memory cards, memory sticks, or smart cards.

As a non-transitory computer-readable medium, storage 411, in addition to a number of applications 412, may store instructions thereon in the form of computer-readable program code. The computer-readable program code may include a number of modules which may, for example, be parts of an individualized liquid feeding software product 413 associated with the service provided in association with the server.

When the system for individualized liquid feeding is arranged such that its functionality is implemented at least partly in the computing device 410 the code may be downloaded from a software application store, for example, from an “App store”, to computing device 410.

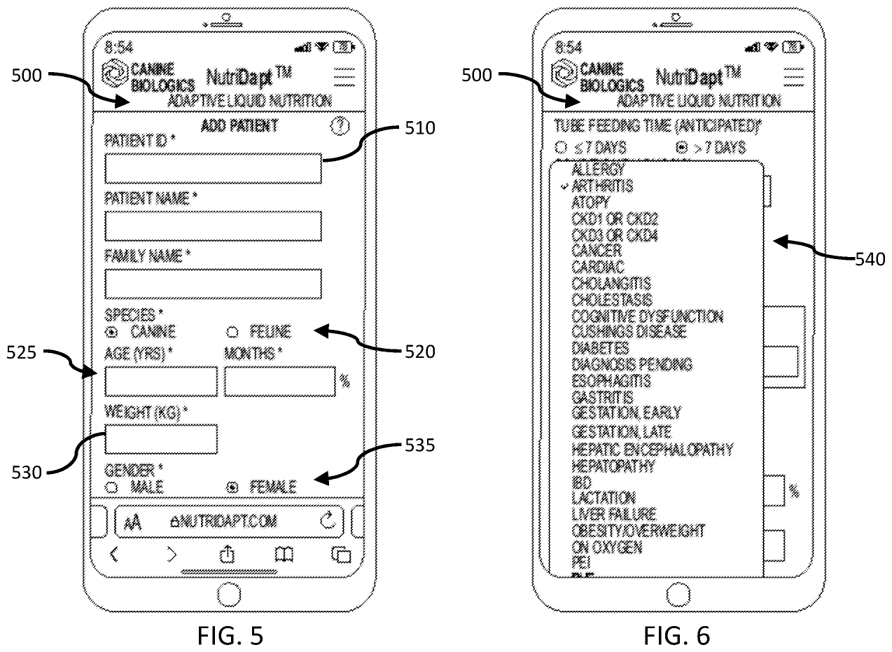

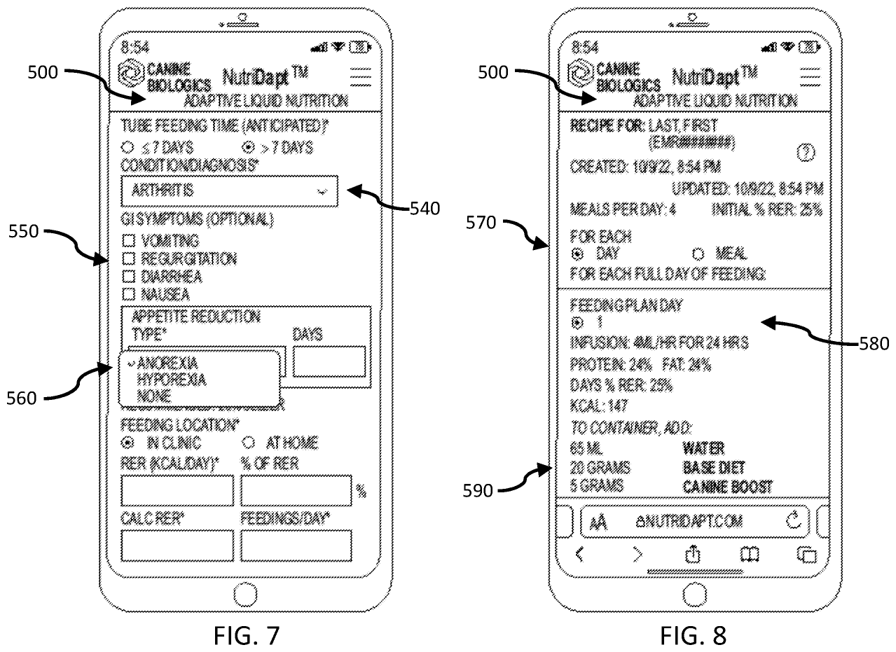

In addition to other actions, executing the software on a processor such as 414 results in generating and rendering a graphical user interface 500 (FIGS. 5-8) on the display screen. Graphical user interface 500 is configured to facilitate user interactions with the individualized liquid feeding service (FIGS. 5-7) for example with tactile input. Graphical user interface 500 includes a number of input windows including windows 510 for inputting identifying information of a patient or other individual for whom a feeding formula is being prepared.

In accordance with the stored code, computing device 410 is configured to, based at least upon an input mass of a particular individual, compute an amount of the powdered, low protein, low fat food suitable for the individual. The input mass may be obtained by a user entering a mass in response to computing device 410 outputting a prompt 530 to input a mass of a particular individual.

The code may cause computing device 410 to compute a daily amount of the powdered, low protein, low fat food based upon an input species of the individual. The species may be obtained by a user entering a species in response to computing device 410 outputting a prompt 520 to input a species of the individual.

The amount computed may be a daily amount. In an example, the amount computed is a resting energy requirement for a canine which is the result of finding the product of 70 and the body mass of the individual in kg to the power 0.75. Thus, an individual canine having 20.45 kg of body mass requires 9.6166×70 or 673.16 kcal of energy at rest. In another example, the amount computed is a resting energy requirement for a feline which is the result of finding the product of 70 and the body mass of the individual in kg to the power 0.67. Thus, an individual feline having 6.5 kg of body mass requires 3.5047×70 or 245.33 kcal of energy at rest.

The code is also configured to cause the computing device 410 to, based at least in part upon a primary health condition, compute an amount of a supplemental powdered protein to be combined with the amount of the powdered, low protein, low fat food as well as an amount of a supplemental nutritional oil to be combined with the amount of the powdered, low protein, low fat food.

The input primary health condition may be obtained by a user entering a condition in response to computing device 410 outputting a prompt 540 to input a primary health condition of a particular individual. In an example, the prompt includes a drop-down list of health conditions.

The code may also be configured to cause computing device 410 to, based upon a viscosity or consistency suitable for liquid feeding, compute an amount of water to be combined with the amount of the powdered, low protein, low fat food, the amount of the supplemental powdered protein and the amount of the nutritional oil.

The code may cause computing device 410 to compute the amount of the supplemental powdered protein based upon input gastrointestinal symptoms of the individual. The code may cause computing device 410 to compute the amount of the nutritional oil based, in part, upon input gastrointestinal symptoms of the individual. The gastrointestinal symptoms may be obtained by a user entering one or more symptoms in response to computing device 410 outputting a prompt 550 to input gastrointestinal symptoms of the individual.

The code may cause computing device 410 to compute the daily amount of the powdered, low protein, low fat food based at least in part upon input appetite. The code may cause computing device 410 to compute the daily amount of the powdered, low protein, low fat food as a series of daily amounts based, in part, upon the input appetite level such as anorexia/anorexic or hyporexia/hyporexic. In an example, the series of daily amounts is an increasing series. In a further example, the amount for a 1^{st} day is 25% of the daily amount, the amount for a 2^{nd} day is 50% of the daily amount, the amount for a 3^{rd} day is 75% of the daily amount and the amount for a 4^{th} day is 100% of the daily amount.

The appetite may be obtained by a user entering an appetite level in response to computing device 410 outputting a prompt 560 to input an appetite of the individual.

The code may cause computing device 410 to compute the amount of the powdered, low protein, low fat food based upon an input age of the particular individual. The age may be obtained by a user entering an age in response to computing device 410 outputting a prompt 525 to input a breed of the individual.

The code may cause computing device 410 to compute the amount of the powdered, low protein, low fat food based at least in part an input gender of the particular individual. The gender may be obtained by a user entering a gender in response to computing device 410 outputting a prompt 535 to input a gender of the individual.

The code may cause computing device 410 to compute the amount of the powdered, low protein, low fat food based upon an input body condition of the particular individual. The body condition may be obtained by a user entering a body condition score in response to computing device 410 outputting a prompt to input a body condition of the individual.

The code may cause computing device 410 to compute the amount of the powdered, low protein, low fat food based at least upon an energy level of the particular individual. The energy level may be obtained by a user entering an energy level in response to computing device 410 outputting a prompt to input an energy level of the individual.

The code may also be configured to cause computing device 410 to output the computed amount of the powdered, low protein, low fat food, the computed amount of the supplemental powdered protein, the computed amount of the supplemental nutritional oil and the computed amount of water (FIG. 8). One or more buttons 570 or other input devices may enable a user to select whether output feeding information is for a single meal or an entire day.

Computed amounts may be output to a portion 590 of graphical user interface 500 in association with an identifier 580 for which day of the plan is being displayed. Example values for the computed amounts of the supplemental powdered protein and the supplemental nutritional oil include zero or none. The code may further cause the computing device 410 to output an analysis of food resulting from combining computed amounts of the formula ingredient.

Referring again to FIG. 4, sensors 419 may include one or more of: an accelerometer, a magnetometer, a pressure sensor, a temperature sensor, a gyroscopic sensor, a Global Positioning System (GPS) sensor, or a timer. The sensors 419 may be used to measure and collect data related to surroundings of the user and or the individual(s).

In some examples, the software product in the form of the stored modules may be interfaced with sensors 419. When executed on processor 414, the software product is configured to resolve and integrate the outputs of sensors 419 into useful information about, for example, the location of computing device 410. In some examples, sensors 419 may include a timer for including time-stamps with computing device locations. Alternatively, processor 414 may provide system time as reference for including the time-stamps with the locations.

Moreover, storage 411 is a non-transient data storage medium. The software product, when executed on the processor 414, is optionally coupled to the storage 411, and is configured to substantially continuously record and update information in storage 411. The information may, for example, include input information about the particular individual or individuals and computing device or module settings.

Network interface 417 optionally allows computing device 410 to upload input information about the particular individual or individuals to a server, for example, via a communication network. Additionally, network interface 417 optionally allows computing device 410 to download information such as computed ratios between the powdered, low fat, low protein food and the powdered protein and the nutritional oil. Furthermore, network interface 417 may allow computing device 410 to access a server to update the software product and/or download one or more new software products associated with the individualized liquid feeding service. Moreover, network interface 417 optionally allows computing device 410 to communicate with other mobile computing devices or PC terminals, for example, via communication network 310.

FIG. 4 is merely an example, which should not unduly limit the scope of the claims herein. It is to be understood that the specific designation for computing device 410 is provided as an example and is not to be construed as limiting the computing device to specific numbers, types, or arrangements of modules and/or components of the computing device. A person skilled in the art will recognize many variations, alternatives, and modifications of embodiments of the disclosure.

Embodiments of the disclosure are susceptible to being used for various purposes, including, though not limited to, enabling users to provide, from two powdered components and a nutritional oil, individualized liquid nutrition that takes disease state(s) and physical characteristics into consideration.

Modifications to embodiments of the disclosure described in the foregoing are possible without departing from the scope of the disclosure as defined by the accompanying claims. Expressions such as “including”, “comprising”, “incorporating”, “consisting of”, “have”, “is” used to describe and claim disclosed features are intended to be construed in a non-exclusive manner, namely allowing for items, components or elements not explicitly described also to be present. Reference to the singular is also to be construed to relate to the plural.

What is claimed is:

1. A system for individualized liquid feeding, comprising:
a powdered, low fat, low protein food fortified with vitamins and minerals;
a powdered protein;
a nutritional oil; and
a computer program product for individualized liquid feeding including a non-transitory computer-readable storage medium storing computer-executable code for outputting liquid feeding instructions, wherein the code, when executed, is configured to cause a computing device to:
based, in part, upon an input mass of an individual, compute a suitable daily amount of the powdered, low protein, low fat food;
based, in part, upon an input primary health condition, compute an amount of the supplemental powdered protein to be combined with the amount of the powdered, low protein, low fat food;
based, in part, upon the input primary health condition, compute an amount of the nutritional oil to be combined with the amount of the powdered, low protein, low fat food; and
output the computed amount of the powdered, low protein, low fat food, the computed amount of the supplemental powdered protein and the amount of the nutritional oil.

2. The computer program product as set forth in claim 1, wherein the code, when executed, is further configured to cause a computing device to output a prompt to input a mass of the individual.

3. The system as set forth in claim 1, wherein the code, when executed, is further configured to cause a computing device to output a prompt to input a primary health condition of the individual.

4. The system as set forth in claim 1, wherein the code is further configured to cause a computing device to, based in part upon a viscosity suitable for liquid feeding, compute an amount of water to be combined with the powdered, low protein, low fat food, the supplemental powdered protein and the nutritional oil.

5. The system as set forth in claim 1, wherein the code, when executed, is further configured to cause a computing device to output a prompt to input a species of the individual.

6. The system as set forth in claim 1, wherein the code configured to cause a computing device to compute the daily amount of the powdered, low protein, low fat food is further configured to cause a computing device to compute the daily amount of the powdered, low protein, low fat food based upon an input species of the individual.

7. The system as set forth in claim 1, wherein the code is further configured to cause a computing device to output a prompt to input one or more gastrointestinal symptoms.

8. The system as set forth in claim 1, wherein the code configured to cause a computing device to compute the amount of the supplemental powdered protein is further configured to cause a computing device to compute the amount of the the supplemental powdered protein based upon input gastrointestinal symptoms of the individual.

9. The system as set forth in claim 1, wherein the code configured to cause a computing device to compute the amount of the nutritional oil is further configured to cause a computing device to compute the amount of the nutritional oil based, in part, upon input gastrointestinal symptoms of the individual.

10. The system as set forth in claim 1, wherein the code is further configured to cause a computing device to output a prompt to input an appetite level.

11. The system as set forth in claim 1, wherein the code configured to cause a computing device to compute the daily amount of the powdered, low protein, low fat food is further configured to cause the computing device to compute the daily amount of the powdered, low protein, low fat food based, in part, upon input appetite level.

12. The system as set forth in claim 1, wherein the code configured to cause a computing device to compute the daily amount of the powdered, low protein, low fat food based is further configured to cause the computing device to compute a series of daily amounts of the powdered, low protein, low fat food based, in part, upon input appetite level.

13. The system as set forth in claim 12, wherein the code configured to cause a computing device to compute the series of daily amounts of the powdered, low protein, low fat food is further configured to cause the computing device to compute an increasing series of daily amounts of the powdered, low protein, low fat food based, in part, upon the input appetite level.

14. A system for individualized liquid feeding of an individual, comprising:
a sealed vessel containing a powdered, low fat, low protein food fortified with vitamins and minerals;
a sealed vessel containing a powdered protein;
a sealed vessel containing a nutritional oil; and
instructions for mixing a portion of the powdered, low fat, low protein food with a portion of the powdered protein and a portion of the nutritional oil in accordance with a mass and primary health condition of the individual.

15. The system as set forth in claim 14, wherein the instructions comprise graphical user interface output.

16. A method for individualized liquid feeding of an individual, comprising:
providing a sealed container of a powdered, low fat, low protein food fortified with vitamins and minerals together with a sealed container of a powdered protein together with a sealed container of a nutritional oil; and
providing instruction to combine a portion of the powdered, low fat, low protein food with a portion of the powdered protein and a portion of the nutritional oil in accordance with a mass and primary health condition of the individual.

17. The method as set forth in claim 16, wherein providing instruction to combine the portion of the powdered, low fat, low protein food with the portion of the powdered protein and the portion of the nutritional oil in accordance with a mass and primary health condition of the individual further comprises computing the portion of the powdered, low fat, low protein food based, in part, upon the mass of the individual.

18. The method as set forth in claim 16, wherein providing instruction to combine the portion of the powdered, low fat, low protein food with the portion of the powdered protein and the portion of the nutritional oil in accordance with a mass and primary health condition of the individual further comprises computing the portion of the powdered protein and the portion of the nutritional oil based, in part, upon the primary health condition of the individual.

19. The method as set forth in claim 16, wherein providing instruction to combine the portion of the powdered, low fat, low protein food with the portion of the powdered protein and the portion of the nutritional oil in accordance with a mass and primary health condition of the individual further comprises computing the portion of the powdered, low fat, low protein food based, in part, upon an appetite level of the individual.

20. The method as set forth in claim 16, wherein providing instruction further comprises outputting to a graphical user interface of a computing device.
